(19) United States
(12) Patent Application Publication
(10) Pub. No.: US 2006/0039938 A1
(43) Pub. Date: Feb. 23, 2006

(54) Cosmetic method of caring for greasy skin

(75) Inventor: Delphine Josse, Gentilly (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/204,158

(22) Filed: Aug. 16, 2005

Related U.S. Application Data
(60) Provisional application No. 60/606,128, filed on Sep. 1, 2004.

(30) Foreign Application Priority Data
Aug. 17, 2004 (FR) 0408937

(51) Int. Cl. A61K 8/81

(52) U.S. Cl. 424/401; 424/70.16

(57) ABSTRACT

The present invention relates to a cosmetic method of caring for greasy skin by topical application to the skin of a composition containing, in a physiologically acceptable medium suitable for topical application to the skin and containing an aqueous phase: (a) porous particles having an average diameter by volume of less than or equal to 10 μm and containing at least one active intended for the treatment of greasy skin and (b) particles of at least one sebum-absorbing and/or sebum-adsorbing compound having a number-average particle size of more than 10 μm.

### REFERENCE TO PRIOR APPLICATIONS

This application claims priority to U.S. provisional application No. 60/606,128 filed Sep. 1, 2004, and to French patent application 0408937 filed Aug. 17, 2004, both incorporated herein by reference.

### FIELD OF THE INVENTION

The present invention relates, in a preferred embodiment, to a cosmetic method of caring for greasy skin, comprising the topical application to the skin of a composition comprising, in a physiologically acceptable medium suitable for topical application to the skin and containing an aqueous phase, (a) porous particles having an average diameter by volume of less than or equal to 10 μm and comprising at least one active intended for the treatment of greasy skin and (b) particles of at least one sebum-absorbing and/or sebum-adsorbing compound having a number-average particle size of more than 10 μm.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Application EP 1 493 433 discloses cosmetic compositions comprising porous particles loaded with cosmetic or dermatological actives intended in particular for the care of greasy skin. The size of these particles is sufficiently small to provide penetration of the active into the skin's pores and hence to transport it to its site of action, in the pilosebaceous unit. Example 1, for instance, discloses a matting powder comprising salicylic acid adsorbed on silica particles and also talc at a level of 69% by weight.

Application FR 04/50887 (unpublished), furthermore, discloses matting compositions comprising sebum-absorbing or sebum-adsorbing compounds in combination with optical-effect fillers such as polyamide (Nylon) microspheres having an average size of 10 μm. There is no suggestion in that application that the nylon microspheres might be loaded with an active.

There remains the need to have compositions available which allow the imperfections of greasy skin to be controlled more effectively than those provided to date in the prior art.

### SUMMARY OF THE INVENTION

The inventor has found that this need is met by combining the aforementioned loaded porous particles with compounds capable of absorbing and/or adsorbing sebum in a composition comprising an aqueous phase. The composition thus obtained allows the matt appearance of the skin to be improved in a lasting fashion. While not bound by any particular theory, it is believed that the reason for this is that the active-loaded porous particles, by removing encrustation from the pilosebaceous canal, in a first phase induce an increase in sebum production at the surface of the skin, which can be captured by the sebum-absorbing and/or sebum-adsorbing fillers. The skin hence remains durably matted.

It is indeed the case that Applications WO 96/17585 and FR-2 657 255 disclose anhydrous cosmetic compositions comprising optionally coated silica particles on which or in which are adsorbed actives and combined with which there may be particles for filling the function of sebum pumps, such as talc or agglomerates of crosslinked polymer (Polytrap®). In these documents, the examples of compositions containing these two types of particles do not include any active intended for the treatment of greasy skin.

Also known, from Application US 2003/032680, is an O/W emulsion containing hydroquinone impregnated on porous microparticles and absorbed into microagglomerates of more than 10 μm (Polytrap®). This emulsion may include various actives—including antioxidants—in combination with the hydroquinone. This emulsion is not intended for the care of greasy skin, but rather for lightening of the skin.

Also known, from Application FR-2 857 254, are compositions in which an optical brightener is impregnated in porous mineral particles of less than 50 μm, preferably of less than 10 μm. This composition may be intended for greasy-skincare and may include a sebum pump. There is no provision that the particles might be impregnated with a active intended for the treatment of greasy skin.

Also known, from Application WO 05/011622, are non-spherical silica-based porous particles on which is adsorbed an optically active substance and with which there may be combined a spherical powder of 0.2 to 20 μm such as nylon. There again, there is no provision that the particles might be impregnated with a active intended for the treatment of greasy skin.

### DETAILED DESCRIPTION OF THE PREFERRED EMBODIMENTS

The present invention accordingly provides a cosmetic method of caring for greasy skin, comprising the topical application to the skin of a composition comprising, in a physiologically acceptable medium suitable for topical application to the skin and comprising an aqueous phase: (a) porous particles having an average diameter by volume of less than or equal to 10 μm and comprising at least one active intended for the treatment of greasy skin and (b) particles of at least one sebum-absorbing and/or sebum-adsorbing compound having a number-average particle size of more than 10 μm.

The constituents of the composition according to the invention will now be described in more detail.

Porous Particles

The term “porous particles” refers to particles having a structure comprising pores. This porous structure may in particular make it possible at least partly to incorporate one or more active agents within said particles.

This structure may be of matrix type, patterned on a sponge. It may also be of vesicular type, which is to say that the particle exhibits an internal cavity delimited by a porous wall.

The porosity associated with the particle size is characterized quantitatively by the particles' specific surface area. The porous particles of the invention exhibit a specific surface area as measured according to the BET method of greater than or equal to 1 m²/g.

The BET (Brunauer-Emmet-Teller) method is a method which is well known to the skilled worker. It is described in particular in “The Journal of the American Chemical Society”, Vol. 60, page 309, February 1938 and corresponds to International Standard ISO 5794/1 (Annex D). The specific surface area as measured according to the BET method corresponds to the total specific surface area, in other words that including the surface formed by the pores.

According to one particular embodiment the particles of the invention exhibit a specific surface area as measured by BET ranging in particular from 2.5 to 1000 m²/g, especially from 3 to 750 m²/g, more particularly greater than or equal to 300 m²/g or even greater than or equal to 500 m²/g.

The porous particles according to the invention are generally individualized particles. The term “individualized particles” refers to particles which are not grouped in aggregate or agglomerate form. These particles preferably exhibit a density greater than or equal to 0.15 g/cm³ and especially ranging from 0.2 to 5 g/cm³.

The particles used according to the present invention preferably derive from preformed porous particles: that is, particles which have been formed in the absence of the compound or compounds to be encapsulated.

In the sense of the present invention the term “loaded particles” will be used below to designate the particles according to the invention, so as to distinguish them from the particulate material from which they derive, which contains no active compound.

As mentioned above, the particles according to the present invention have a volume-average diameter of less than or equal to 10 μm. This is because particles of this kind are able to penetrate the sebaceous follicle by application of a mechanical force. This mechanical force originates generally from a massage which, in addition to the thrust that it exerts, generates a pump effect at the level of the follicle. The particles thus progressively reach the follicular canal, in which the active compound they carry may then diffuse, and may optionally enter the tissues surrounding the follicular canal. In contrast the support, as constituted by the particles, is subsequently rejected by virtue of the flow of sebum and/or of the growth of the hair, thereby making it possible to prevent any undesirable reaction of the body with the solid compound constituting the particles.

It should be noted that particles having a diameter of more than 10 μm, even with application of a similar mechanical force, remain for the most part localized on the skin's surface, without penetrating it, and are therefore unable to release the active compound anywhere other than on the horny layer.

According to one particular embodiment of the invention the particles have a volume-average diameter of greater than or equal to 0.1 μm and in particular range from 0.5 to 8 μm, including 0.2, 0.3, 0.4, 0.6, 0.8, 1, 1.5, 2, 2.5, 3, 3.5, 4, 4.5, 5, 5.5, 6, 6.5, 7, and 7.5 μm.

The particles used according to the invention are preferably particles, especially spherical particles, which are porous and have a number-average size which can range from 0.1 to 50 μm, especially from 0.1 to 20 μm and very particularly from 0.5 to 10 μm.

According to one version of the invention the particles are characterized by their granulometric homogeneity. In particular they exhibit a polydispersity index, PI, ranging from 1 to 4 and especially less than or equal to 3. This polydispersity index is defined as the ratio D(4.3)/D(3.2), in which D(4.3) denotes the volume-average diameter and D(3.2) denotes the surface-average diameter. These two values are commonly measured by means of laser-diffraction particle-size analysers like that sold under the name “Mastersizer 2000”, by Malvern.

The porous particles of the invention may have various forms, especially globular and in particular substantially spherical.

These porous particles are generally preferably composed of materials which are completely insensitive, in terms in particular of solubilization and plastification, to the process of encapsulation of the active compounds, especially where said process involves an organic solvent for the impregnation.

These particles may be of organic, inorganic or hybrid type and most often are in the form of a powder having, in particular, a reduced volatility.

As organic porous particles mention may be made, by way of example, of polyamide particles, especially particles of Nylon 6, Nylon 6-6, Nylon 12 or Nylon 6-12, like those sold by Atofina under the generic name “Orgasol”, and polymethyl methacrylate (PMMA) particles like those sold under the name “Covabead®” by Wacker.

In one particular embodiment of the invention the particles used are selected from polyamide (Nylon®) particles like those mentioned above.

According to one version of the invention the particles according to the invention are inorganic in nature.

By way of illustration of inorganic materials which can be used in the particles according to the invention mention may be made of silica, silica-alumina composites, hydroxyapatite, titanium dioxide, sericite, mica, magnesium carbonate or hydrogencarbonate, aluminium oxides of alumina type, and mixed silicates, such as aluminosilicates, and mixtures thereof.

In particular the porous inorganic particles suitable for the invention may be selected from the following:

silica particles like those sold by Asahi Glass under the name “Sunsphere H series”, and by Suzuki Oil and Fat under the name “God Balls”,

hydroxyapatite particles like those sold by Merck (under reference 1051990010—particle size 15 μm) or else those sold by Laboratory Skin Care and Sekisui under the respective names “Hydroxyzomes” (LCS), and AP20C and AP12C (Sekisui), “ASP®” by Sekisui Plastics, or under the name “Hydroxyzomes” by Asahi Glass,

silica-alumina particles like those sold under the name “Zeeosphere®” by 3M,

titanium dioxide particles like those sold by Ishihara, and

particles composed of a mixture of these minerals.

In one embodiment of the invention the particles used are selected in particular from particles of silica and of hydroxyapatite.

The porous particles used in the present invention may also be composed of composite organic and inorganic materials.

Mixtures of various porous particles may be used, of course.

The loaded particles of the present invention can be prepared according to conventional methods, in particular by impregnation. In particular the loaded particles according to the invention are obtained by impregnating preformed porous particles with at least one active compound. Advantageously this protocol does not require the presence of a pore former.

By way of example, the porous particles containing the active are obtainable according to an impregnation process comprising the following steps:

dissolving the active to be encapsulated in a solvent such as acetone or ethanol to give an impregnating solution,
impregnating the porous particles with said impregnating solution,
evaporating the solvent until a dry powder is obtained.

The powder thus obtained generally contains only a very low proportion of residual solvent, of the order of 1/10 ppm.

As solvents which can be used in such an impregnating process mention may be made in particular of acetone, ethanol, isopropanol, dichloromethane and ethyl acetate. It will be appreciated that the selection of the solvent is made taking into account the nature of the components of the porous particles and the compounds to be encapsulated.

Generally the composition contains from 0.1% to 50% by weight, preferably from 0.2% to 20% by weight and more preferentially from 1% to 5% by weight of loaded porous particles, relative to the total weight of the composition.

Active Intended for the Treatment of Greasy Skin

The term “active intended for the treatment of greasy skin” refers, in the context of the present invention, to a compound which itself—that is, not necessitating the involvement of an external agent in order to activate it—has an activity which may be in particular:

a desquamating activity (allowing the opening up of comedos), and/or

an antimicrobial activity (especially on P. acnes), and/or

an anti-inflammatory activity, and/or

a seboregulatory activity, and/or

an antioxidant activity (preventing the oxidation of squalene and the formation of comedos).

The active intended for the treatment of greasy skin may therefore be preferably selected from the following: desquamating and/or antimicrobial and/or anti-inflammatory and/or seboregulatory and/or antioxidant agents.

1. Desquamating Agents

A “desquamating agent” is any compound capable of acting:

either directly on desquamation, by promoting exfoliation, such as β-hydroxy acids, especially salicylic acid and its derivatives (including 5-(n-octanoyl)salicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol and certain jasmonic acid derivatives;

or on enzymes involved in the desquamation or the breakdown of corneodesmosomes, glycosidases, stratum corneum chymotryptic enzyme (SCCE) or even other proteases (trypsin, chymotrypsin-like proteases). Mention may be made of amino sulphonic compounds and in particular (N-2-hydroxyethylpiperazine-N-2-ethane)-sulphonic acid (HEPES); derivatives of 2-oxothiazolidine-4-carboxylic acid (procysteine); derivatives of alpha-amino acids of glycine type (as described in EP 0 852 949, and also the sodium methylglycine diacetate sold by BASF under the trade name Trilon M); honey; and sugar derivatives such as O-octanoyl-6-D-maltose and N-acetylglucosamine.

5-(n-Octanoyl)salicylic acid is preferred for use in the present invention.

2. Antimicrobial Agents

Antimicrobial agents which can be used in the composition according to the invention include those which may be selected in particular from 2,4,4′-trichloro-2′-hydroxydiphenyl ether (or triclosan), 3,4,4′-trichlorocarbanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopiroxe, ciclopiroxol-amine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, octopirox, octoxyglycerol, octanolglycine, caprylyl glycol, 10-hydroxy-2-decanoic acid, dichlorophenyl imidazole dioxolane and its derivatives described in Patent WO9318743, copper pidolate, salicylic acid, iodopropynyl butylcarbamate, farnesol, phytosphingosines and mixtures thereof.

Preferred antimicrobial agents are octoxyglycerol, copper pidolate, salicylic acid and iodopropynyl butylcarbamate.

3. Anti-Inflammatory Agents

Useful anti-inflammatory or calmative agents which can be used in the composition according to the invention include the following: pentacyclic triterpenes and plant extracts (e.g. Glycyrrhiza glabra) containing them, such as β-glycyrrhetinic acid and its salts and/or its derivatives (glycyrrhetinic acid monoglucuronide, stearyl glycyrrhetinate, 3-stearoyloxy glycyrrhetic acid), ursolic acid and its salts, oleanolic acid and its salts, betulinic acid and its salts, bisabolol, an extract of Paeonia suffruticosa and/or lactiflora, salts of salicylic acid, and in particular zinc salicylate, phycosaccharides from Codif, an extract of Laminaria saccharina, canola oil, bisabolol and camomile extracts, allantoin, Sepivital EPC (phosphoric diester of vitamin E and C) from Seppic, omega-3 unsaturated oils such as muscat rose oil, blackcurrant oil, echium oil, fish oil, plankton extracts, capryloyl glycine, Seppicalm VG (sodium palmitoylproline and Nymphea alba) from Seppic, an extract of pygeum, an extract of Boswellia serrata, an extract of Centipeda cunninghami, an extract of Helianthus annuus, an extract of Linum usitatissimum, tocotrienols, extracts of Cola nitida, extracts of Centella asiatica, piperonal, an extract of clove, an extract of Epilobium angustifolium, aloe vera, an extract of Bacopa monieri, phytosterols, niacinamide, cortisone, hydrocortisone, indomethacin and beta-methasone.

Anti-inflammatory agents preferred for use in the present invention are extracts of Centella asiatica, β-glycyrrhetinic acid and its salts, alpha-bisabolol and niacinamide.

4. Seboregulatory Agents

When the composition according to the invention comprises a seboregulatory agent such as a 5α-reductase inhibitor, said agent may preferably be selected from the following:

retinoids, and particularly retinol;

sulphur and sulphur-containing derivatives;

zinc salts such as zinc lactate, zinc gluconate, zinc pidolate, zinc carboxylate, zinc salicylate and/or zinc cysteate;

selenium chloride;

vitamin B6 or pyridoxine;

the mixture of capryloylglycine, sarcosine and extract of Cinnamomum zeylanicum sold in particular by Seppic under the trade name Sepicontrol A5®;

an extract of Laminaria saccharina sold in particular by Secma under the trade name Phlorogine®;

an extract of Spiraea ulmaria sold in particular by Silab under the trade name Sebonormine®;

plant extracts of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all sold, for example, by Maruzen;

an extract of Serenoa repens, sold in particular by Euromed;

plant extracts of the genus Silybum;

plant extracts containing sapogenins, and especially the diosgenin-rich extracts of Dioscoreaceae; and

extracts of Eugenia caryophyllata containing eugenol and eugenyl glucoside.

Zinc salts are preferred for use in the present invention.

5. Antioxidant Agents

Preferred antioxidant agents for use in the present invention include those selected from tocopherol and its esters, such as tocopherol acetate; BHT and BHA.

The active or actives used in the composition according to the invention may represent from 1% to 50%, preferably from 2% to 40% and better still from 5% to 30% of the total weight of the loaded particles. More than one active may be present in any given particle, and mixtures of loaded particles, each with one of more unique active therein, may be used.

Sebum-Absorbing and/or Sebum-Adsorbing Compound

A “sebum-absorbing and/or sebum-adsorbing compound” is a compound capable of absorbing and/or adsorbing sebum, commonly designated by “sebum pump”.

Generally speaking, this type of compound is in the form of a powder of particles which take up sebum. The sebum uptake of these compounds is advantageously greater than or equal to 1 ml/g, especially greater than or equal to 2 ml/g and in particular greater than or equal to 3 ml/g. The sebum uptake corresponds to the amount of sebum absorbed and/or adsorbed on the available surface of the particles.

It is measured according to the so-called Wet Point method or powder oil uptake determination method described in the Standard NF T 30-022. It corresponds to the amount of sebum adsorbed on the available surface of the powder and/or absorbed by the powder by measurement of the Wet Point, described below:

A quantity m (in grams) of powder, of between approximately 0.5 g and 5 g (the amount depends on the density of the powder), is placed on a glass plate and then artificial sebum is added dropwise, this sebum being maintained at a temperature of 29° C. and having the following composition, expressed in % by weight:

| | |
|---|---|
| triolein | 29.00% |
| oleic acid | 28.50% |
| oleyl oleate | 18.50% |
| squalene | 14.00% |
| cholesterol | 7.00% |
| cholesterol palmitate | 3.00% |

Following the addition of 4 to 5 drops of artificial sebum, the artificial sebum is incorporated into the powder by means of a spatula, and the addition of the artificial sebum is continued until conglomerates of artificial sebum and powder are formed. From this point onwards, the artificial sebum is added one drop at a time and thereafter the mixture is triturated with the spatula. The addition of artificial sebum is halted when a firm, smooth paste is obtained. This paste must be able to be spread over the glass plate without cracks or formation of crumbs. The volume Vs (expressed in ml) of artificial sebum used is then recorded.

The sebum uptake corresponds to the ratio Vs/m.

The particles of sebum-absorbing and/or sebum-adsorbing compound preferably exhibit an average size of more than 10 μm, so as not to enter into competition with the porous/loaded particles described above.

According to one particular embodiment the particles of sebum-absorbing and/or sebum-adsorbing compound may exhibit a BET specific surface area of greater than or equal to 300 m²/g, especially greater than or equal to 500 m²/g and in particular greater than or equal to 600 m²/g, and especially less than or equal to 1500 m²/g.

The particles of sebum-absorbing and/or sebum-adsorbing compound may preferably be mineral or organic in origin, more particularly organic in origin. More specifically this compound may be selected from powders of polyamide (Nylon®), powders of acrylic polymers, especially of polymethyl methacrylate/ethylene glycol dimethacrylate, of polyallyl methacrylate/ethylene glycol dimethacrylate, and of ethylene glycol dimethacrylate/lauryl methacrylate copolymer, powders of crosslinked polyalkylstyrene, and mixtures thereof.

The particles of this compound may where appropriate be surface-treated with at least one hydrophobic treatment agent, in particular a non-fluorinated agent.

This hydrophobic treatment agent may be selected in particular from:

silicones, such as methicones and dimethicones;

fatty acids, such as stearic acid;

metal soaps, such as aluminium dimyristate and the aluminium salt of hydrogenated tallow glutamate;

amino acids, N-acylamino acids of salts thereof;

lecithin, isopropyl triisostearyl titanate; and

mixtures thereof.

The N-acylamino acids may contain an acyl group containing 8 to 22 carbon atoms, such as, for example, a 2-ethylhexanoyl, caproyl, lauroyl, myristoyl, palmitoyl, stearoyl or cocoyl group. The salts of these compounds may be the aluminium, magnesium, calcium, zirconium, zinc, sodium or potassium salts. The amino acid may be, for example, lysine, glutamic acid or alanine.

AS non-limitative representatives of the sebum-absorbing and/or sebum-adsorbing compounds according to the invention, mention may be made very particularly of the following:

powders of polyamides (Nylon®), such as, for example, “Orgasol® 2002 D NAT COS” sold by Atofina,

powders of acrylic polymers, particularly of polymethyl methacrylate/ethylene glycol dimethacrylate, such as, for example, “Dow Corning 5640 Microsponge® Skin Oil Adsorber” sold by Dow Corning, or “Micropearl M 305” sold by Matsumoto Yushi; powders of polyallyl methacrylate/ethylene glycol dimethacrylate, such as, for example, “Poly-Pore® E200” sold by Amcol; powders of ethylene glycol dimethacrylate/lauryl methacrylate copolymer, such as, for example, “Polytrap® 6603” sold by RP Scherer, and

powders of crosslinked polyalkylstyrene, such as the “Imbiber Beads 295” sold by Imbibitive Technologies.

The sebum-absorbing and/or sebum-adsorbing compound preferably represents from 0.1% to 10% by weight and preferably from 1% to 5% by weight relative to the total weight of the composition. Mixtures of such compounds may be used, of course.

The composition according to the invention is preferably generally suitable for topical application to the skin and therefore generally comprises a physiologically acceptable medium, i.e. a medium which is compatible with the skin and/or its epidermal derivatives. The medium in question is preferably cosmetically acceptable, which is to say that it has a colour, odour and feel which are agreeable and does not give rise to unacceptable discomfort (stinging, tautening, redness) liable to put the consumer off using this composition.

The composition according to the invention may be in any form, including all of the formulating forms conventionally used for topical application, and especially in the form of dispersions of lotion or gel type, of emulsions of liquid or semi-liquid consistency of the milk type, obtained by dispersing a fatty phase in an aqueous phase (O/W) or conversely (W/O), or of suspensions or emulsions of soft, semi-solid or solid consistency, of the cream or gel type, or else of multiple emulsions (W/O/W or O/W/O), of microemulsions, of vesicle dispersions of ionic and/or nonionic type, or of wax/aqueous phase dispersions. These compositions may be prepared according to customary methods.

According to one preferred embodiment of the invention the composition is in the form of an O/W emulsion.

The composition according to the invention may be a composition for caring for, cleansing or making up the skin of the body or face.

This composition may further comprise various adjuvants used in the cosmetic field, such as emulsifiers, including glyceryl fatty acid esters, sugar fatty acid esters, optionally polyoxyethylenated, polyoxyethylene fatty acid esters, sorbitan fatty acid esters; fillers, especially soft-focus effect fillers such as colloidal dispersions of silica or of composite silica-alumina filler or else polyamide fibres; preservatives; sequestrants; colorants; perfumes; and thickeners and gelling agents, especially acrylamide homo- and copolymers, acrylic homo- and copolymers and acrylamidomethylpropanesulphonic acid (AMPS) homo- and copolymers.

It may also contain actives other than that included in the porous particles used according to the invention. These additional actives may be selected for example from the desquamating, antimicrobial, calmative and seboregulatory agents described above.

The person skilled in the art will of course take care to select this or these optional additional compounds and/or their amount in such a way that the advantageous properties of the composition according to the invention are not, or not substantially, adversely affected by the intended addition.

The invention will now be illustrated by the following non-limitative example.

### EXAMPLE

### Fluid for Greasy Skin

a) Preparation of Loaded Porous Particles

0.5 g of tocopherol and 1 g of 5-(n-octanoyl)salicylic acid are dissolved in acetone. Into this mixture is introduced 13.5 g of porous particles of Nylon-12, sold under the name “Orgasol 2002 UD NAT COS” by Atofina. The dispersion is subsequently introduced into a rotary evaporator in order to remove the acetone. This gives a powder loaded with tocopherol and salicylic acid derivative.

b) Preparation of the Cosmetic Composition

An O/W emulsion having the composition below is prepared.

| A | Water | | 61% |
|---|---|---|---|
| | Glycerol | | 5% |
| | Preservatives | | 0.5% |
| B | Polyoxyethylene (100 EO) glyceryl stearate | | 2% |
| | Cetyl alcohol | | 1% |
| | Polysorbate-20 | | 2% |
| | Preservatives | | 0.2% |
| | Isononyl isononanoate | | 6% |
| | Octyldodecanol | | 4% |
| | 5-(n-Octanoyl) salicylic acid | | 0.5% |
| C | Cyclohexasiloxane | | 5% |
| | Xanthan gum | | 0.2% |
| | Acrylic copolymer | | 0.45% |
| | (Pemulen TR2 from Noveon) | | |
| D | Water | qs for | 100% |
| | Sodium hydroxide solution | qs for | pH 5 |

| | | |
|---|---|---|
| E | Loaded nylon particles | 3% |
| F | Ethylene glycol dimethacrylate/lauryl | 1% |
| | methacrylate copolymer* | |

*“Polytrap ® 6603” sold by R P Scherer

Procedure

Phase A is heated with stirring at 80° C. until completely dissolved and then the temperature is brought to 70° C. Phase B is heated with stirring at 70° C. until a clear phase is obtained, and then added to phase A with stirring. The xanthan gum and the acrylic copolymer are dispersed in the cyclohexasiloxane at ambient temperature for 10 minutes and phase C is added to the mixture A+B. Then phase D, dissolved beforehand, is added to the mixture thus obtained. The mixture A+B+C+D is subsequently cooled to 25° C. Phases E and F are dispersed in succession in the mixture obtained beforehand.

This composition may be applied to the face morning and/or evening in order to make greasy skin with a tendency towards acne matt.

The above written description of the invention provides a manner and process of making and using it such that any person skilled in this art is enabled to make and use the same, this enablement being provided in particular for the subject matter of the appended claims, which make up a part of the original description and including method of caring for greasy skin, comprising topically applying to skin in need thereof a composition comprising, in a physiologically acceptable medium comprising an aqueous phase: (a) porous particles having an average diameter by volume of less than or equal to 10 μm and comprising at least one active for the treatment of greasy skin and (b) particles of at least one sebum-absorbing and/or sebum-adsorbing compound having a number-average particle size of more than 10 μm.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or: range is stated, the endpoints are included. Also, all values and subranges within a numerical limit or range are specifically included as if explicitly written out. Terms such as “contain(s)” and the like as used herein are open terms meaning ‘including at least’ unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method of caring for greasy skin, comprising topically applying to skin in need thereof a composition comprising, in a physiologically acceptable medium comprising an aqueous phase: (a) porous particles having an average diameter by volume of less than or equal to 10 μm and comprising at least one active for the treatment of greasy skin and (b) particles of at least one sebum-absorbing and/or sebum-adsorbing compound having a number-average particle size of more than 10 μm.

2. The method according to claim 1, wherein the porous particles are selected from polyamide particles, polymethyl methacrylate particles, silica particles and hydroxyapatite particles, and mixtures thereof.

3. The method according to claim 1, comprising porous polyamide particles.

4. The method according to claim 1, wherein the porous particles have a volume-average diameter of greater than or equal to 0.1 μm.

5. The method according to claim 1, wherein the composition comprises from 1% to 5% by weight of porous particles, relative to the total weight of the composition.

6. The method according to claim 1, wherein the active is selected from desquamating, antimicrobial, anti-inflammatory, seboregulatory and antioxidant agents, and mixtures thereof.

7. The method according to claim 1, wherein the porous particles comprise 5-(n-octanoyl)salicylic acid.

8. The method according to claim 1, wherein the porous particles comprise at least one of the following: octoxyglycerol, copper pidolate, salicylic acid and iodopropynyl butylcarbamate.

9. The method according to claim 1, wherein the porous particles comprise at least one of the following: extracts of Centella asiatica, β-glycyrrhetinic acid and its salts, alpha-bisabolol and niacinamide.

10. The method according to claim 1, wherein the porous particles comprise at least one zinc salt.

11. The method according to claim 1, wherein the porous particles comprise at least one of the following: tocopherol and tocopherol acetate.

12. Method according to claim 1, wherein the active represents from 5% to 30% of the total weight of the particle/active combination.

13. The method according to claim 1, wherein the porous particles are obtained according to a process comprising:
dissolving the active in a solvent to give an impregnating solution,
impregnating the porous particles with said impregnating solution,
evaporating the solvent until a dry powder is obtained.

14. The method according to claim 1, wherein the sebum-absorbing and/or sebum-adsorbing compound is organic.

15. The method according to claim 1, wherein the sebum-absorbing and/or sebum-adsorbing compound is selected from powders of polyamide; powders of polymethyl methacrylate/ethylene glycol dimethacrylate; powders of polyallyl methacrylate/ethylene glycol dimethacrylate; powders of ethylene glycol dimethacrylate/lauryl methacrylate copolymer; powders of crosslinked polyalkylstyrene; and mixtures thereof.

16. The method according to claim 1, wherein the sebum-absorbing and/or sebum-adsorbing compound represents from 1% to 5% by weight relative to the total weight of the composition.

17. The method according to claim 1, wherein the porous particles have a volume-average diameter of 0.5 to 8 μm and the particles of sebum-absorbing and/or sebum-adsorbing compound exhibit a BET specific surface area of 300 m²/g-1500 m²/g.

18. The method according to claim 17, wherein the composition comprises from 1% to 5% by weight of porous particles, relative to the total weight of the composition, wherein the active represents from 5% to 30% of the total weight of the particle/active combination, and wherein the sebum-absorbing and/or sebum-adsorbing compound represents from 1% to 5% by weight relative to the total weight of the composition.
